(19) United States
(12) Patent Application Publication
(10) Pub. No.: US 2005/0192477 A1
(43) Pub. Date: Sep. 1, 2005

(54) Rigid endoscope optics with a compound housing

(75) Inventors: Thomas Forster, Molln (DE); Mathias Kraas, Haseldorf (DE); Gilbert Spahn, Schwarzenbek (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/054,813

(22) Filed: Feb. 10, 2005

(30) Foreign Application Priority Data
Feb. 26, 2004 (DE) 10 2004 009 383.0

(51) Int. Cl. A61B 1/00

(52) U.S. Cl. 600/133; 600/138

(57) ABSTRACT

A rigid endoscope optics (1) including a housing formed by several housing parts (5, 8, 9, 6, 16) that hermetically enclose optic elements (3, 2, 4) and are fitted with hermetic connections (7, 11, 13, 14, 15) between each other. At least one of the housing parts (8, 9) is made of plastic. At least at one of the connection sites of the plastic housing part (8, 9), the plastic has metal coating (11, 13, 14, 15) and the connection is a solder joint.

### BACKGROUND OF THE INVENTION

Endoscope optics of having several housing parts are protected by a hermetic housing against entry of water, and in particular, also against entry of water vapor, and accordingly can be steam-sterilized before being used in surgery without the steam fogging optical surfaces.

In part, the housing consists of an elongated tube receiving an image guide and being sealed at the distal end by a hermetically sealed window. Housing parts are connected to the proximal tube end and they receive, within an inner space, further devices such as ocular lenses, pertinent, hermetically sealed windows, and the like.

The connections between the housing parts must be hermetic. Therefore, as regards known endoscopes of this type, the housing parts are metallic and welded or soldered to each other. The windows, which are parts of the enclosing housing, also are soldered into place. Effective hermetic junctions can only be made in this manner, namely by welding or soldering, whereas such junctions can be implemented adhesively only with great difficulties, if at all.

This restriction to metals for the housing parts represents a drawback.

### BRIEF SUMMARY OF THE INVENTION

The objective of the present invention is to make possible endoscope optics of the above type while making use of plastic housing parts.

In the present invention, the housing parts may be plastic and still be hermetically joined to the remaining housing parts. This objective is attained by coating a connection site with metal, thus allowing solder to be deposited onto it for hermeticity. As a result, a hermetic housing is feasible using plastic parts, offering lower costs and more latitude in construction.

Advantageously, the plastic is PEEK (polyether-etherketone). This plastic has been found to be practical for years in endoscope engineering using non-hermetic parts and is easily metallized.

### BRIEF DESCRIPTION OF THE DRAWING

FIG. 1 shows the present invention in an illustrative and schematic manner in the form of an axial section of endoscope optics.

### DETAILED DESCRIPTION OF THE INVENTION

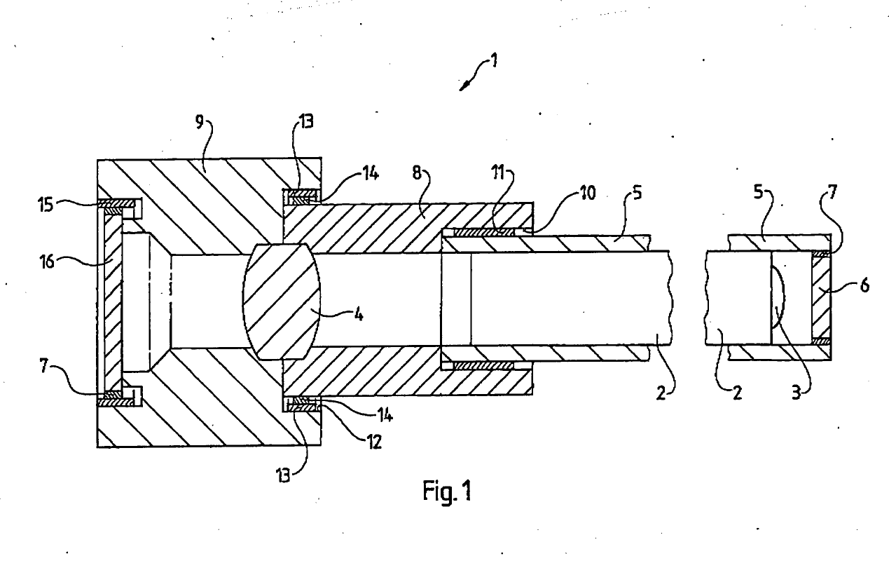

FIG. 1 shows endoscope optics 1 with an elongated image guide 2 having an objective 3 at its distal end. The image guide 2 may be a set of relay lens elements or a bundle of optic fibers, and optionally, also, it may be a video endoscope image guide fitted with a video camera at the objective 3 and with data wires running proximally.

In the shown embodiment, the image guide 2 is an optical image guide and is viewed on the other side of its proximal end through an ocular lens element 4.

The optical system described so far, above, is mounted in a hermetically sealed housing. This housing is fitted at its distal end zone with an elongated tube 5, which for the sake of mechanical strength, is made of metal and which is sealed at its distal end by a window 6. This window is fitted at its rim with a metal coating 7 shown for illustration in a much exaggerated size in the drawing and by means of this coating the window is hermetically soldered to the tube 5.

The tube 5 is affixed to a middle housing part 8 which in turn is affixed in a terminal housing part 9.

The two housing parts 8 and 9 are made of PEEK (polyether-etherketone), a plastic widely used with endoscope optics. At its cylindrical rest surface 10, the housing part 8 comprises a metal coating 11 which is hermetically soldered to the metal tube 5.

In the present embodiment, the housing part 8 is inserted into a borehole 12 of the housing part 9 at the connection site between the housing parts 8 and 9. At that connection site, the borehole 9 comprises a metal coating 13 and the outer surface of the housing part 8 comprises a metal coating 14. Again these two metal coatings are hermetically soldered to each other. All metal coatings shown in FIG. 1 are shown exaggeratedly large.

The terminal housing part 9 is fitted at its proximal end with a metal coating 15 on the inside surface of a terminal borehole. A proximal window 16 is inserted at this site, which is fitted with a metal coating 7 just like the distal window 6. The metal coatings 7 and 15 are hermetically soldered to each other.

As a result, the optic elements 3, 2, 4 are enclosed by a housing which is constituted by the tubular parts 5, 8, 9 and the two terminal windows 6 and 16, and which is soldered to be hermetically sealed at all connection sites.

FIG. 1 is strongly schematic and didactic. For instance, it omits the typical image guides used for illumination, which may be added in known manner to the design shown.

In the embodiment of FIG. 1, the plastic housing parts 8 and 9 are soldered together by means of metal coatings at all connection sites 11, 13, 15. Alternatively, however, the plastic housing parts also may be joined using technically different means. For instance, as regards the embodiment of FIG. 1, the connection at the connection sites 11 and 15 may be implemented by soldering together the metal coatings illustrated in FIG. 1. However, as regards the connection site of the housing parts 8 and 9, another junction mode, free of metal coatings and free of soldering, may be selected. Illustratively, the housing parts may be joined by a direct plastic bond, for instance by fusing the plastic or by a highly sealing adhesive.

1. A rigid endoscope optics (1) comprising a housing, said housing consisting of several housing parts (5, 8, 9, 6, 16) and hermetically enclosing optic elements (3, 2, 4), said housing being fitted with hermetic connections (7, 11, 13, 14, 15) between said housing parts, wherein at least one of the housing parts (8, 9) is made of plastic, at least one connection site of the at least one plastic housing part (8, 9) has a metal coating (11, 13, 14, 15) and the at least one connection site is soldered.

2. The endoscope optics as claimed in claim 1, wherein the plastic is PEEK.
